(19) United States
(12) Patent Application Publication
(10) Pub. No.: US 2017/0326328 A1
(43) Pub. Date: Nov. 16, 2017

(54) PERIPHERAL NEUROPATHY BREATHING DEVICE

(71) Applicants: Joshua Hensley, Alexandria, VA (US); Elijah Hensley, Alexandria, VA (US); Sydney McDermott, Viera, FL (US)

(72) Inventors: Joshua Hensley, Alexandria, VA (US); Elijah Hensley, Alexandria, VA (US); Sydney McDermott, Viera, FL (US)

(21) Appl. No.: 15/151,638

(22) Filed: May 11, 2016

(51) Int. Cl. A61M 16/10; A61M 16/06; A61M 16/00; A61M 16/08

(52) U.S. Cl. CPC A61M 16/1095; A61M 16/0875; A61M 16/107; A61M 16/0057; A61M 16/0666

(57) ABSTRACT

A peripheral neuropathy breathing device for attachment to a person for warming the air breathed by the person thereby reducing the extreme discomfort caused by cold-weather sensitive peripheral neuropathy. The device according to some embodiments employs an air pump energized by a battery. The air pump has an input line disposed to accept air warmed by the body and an output line for bringing the air to a facemask. A patient wearing the facemask breathes warmed air to significantly reduce the discomfort and pain caused by cold-weather sensitive peripheral neuropathy. The design, in employing air warmed by the body of the person using the device, eliminates the need to warm the air thereby minimizing power, battery size, or battery replacement.

### BACKGROUND

A common therapy for cancer patients is chemotherapy. Unfortunately, chemotherapy creates a number of side effects including; nausea, constipation, peripheral neuropathy, and similar effects. The volume and frequency of a particular side effect varies from patient to patient. A side effect can be so problematic that it forces the doctor to reduce or eliminate the chemotherapy regimen. Unfortunately, reducing the chemotherapy regimen can permit the cancer to grow or spread and eventually cause the death of the patient.

Currently, if a patient experiences a particular side effect doctors will often provide medicine or other means to reduce the volume of the side effect such that the patient can withstand the side effect and still receive the planned chemotherapy regimen. For example, nausea is treated with anti-nausea pills.

One side effect—cold weather sensitive peripheral neuropathy affects about 10% of chemotherapy patients depending upon the type of chemotherapy and the patient's characteristics. Problems resulting from cold weather sensitive peripheral neuropathy include a feeling that sharp objects are piercing the fingers when the patient grabs a cold object, e.g., from the refrigerator. In addition, a patient breathing cold or cool air feels sharp objects piercing the interior of the nose and/or trachea. Cold air below 55° F. can cause this problem and the problem intensifies as the temperature drops. A patient can wear a mask or place his hand in front of his nose/mouth; however, this does not acceptably or effectively reduce the suffering. In a cold climate where the temperatures could be in the 30s, the patient could be subjected to substantial pain with each and every breath. This is a very serious and brutal problem which could easily force the patient to cease chemotherapy. Because for most patients chemotherapy is the only therapy, its discontinuance removes any hope of survival.

### DESCRIPTION OF THE DRAWINGS

Aspects of the present disclosure are understood from the following detailed description and when read with the accompanying figures. It is noted that, in accordance with the standard practice in the industry, various features are not drawn to scale. In fact, the dimensions of the various features may be arbitrarily increased or reduced for clarity of discussion. One or more embodiments are illustrated by way of example, and not by limitation, in the figures of the accompanying drawings, wherein elements having the same reference numeral designations represent like elements throughout and wherein:

FIG. 1 is a front view of a person wearing a peripheral neuropathy breathing device according to one or more embodiments.

FIG. 2 is a front view of a peripheral neuropathy breathing device according to one or more embodiments.

FIG. 3 is a front view of an air pump apparatus usable in conjunction with the peripheral neuropathy breathing device of FIG. 2 according to one or more embodiments.

FIG. 4 is a front view of a facemask usable in conjunction with the peripheral neuropathy breathing device FIG. 2 according to one or more embodiments.

FIG. 5 is a front view of a person wearing a covering according to one or more embodiments.

FIG. 6 is a front view of a person wearing nasal air tube 22 replacing the facemask and without the covering according to one or more embodiments.

### DETAILED DESCRIPTION

The following disclosure provides many different embodiments, or examples, for implementing different features of the invention. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

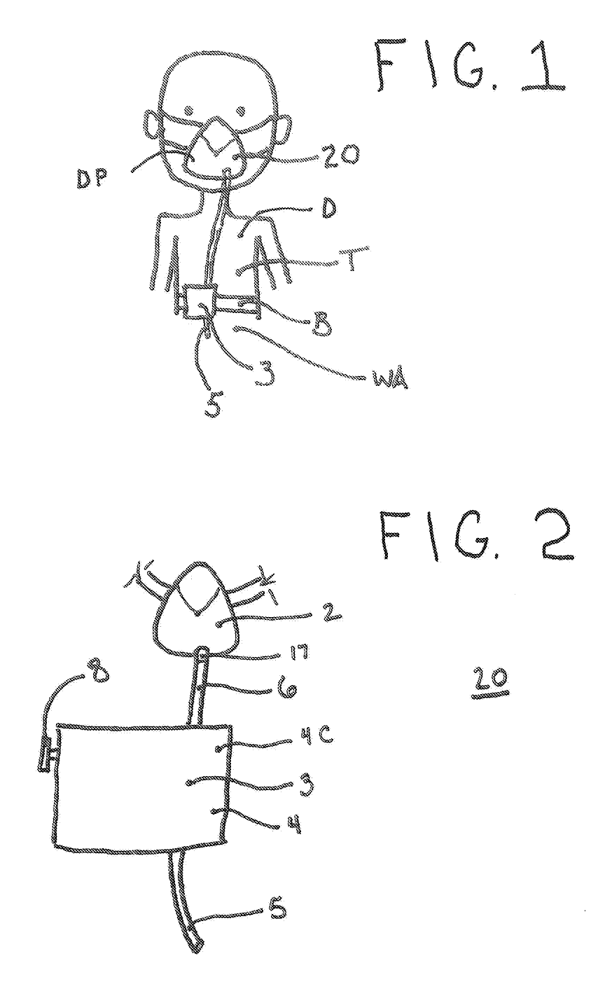

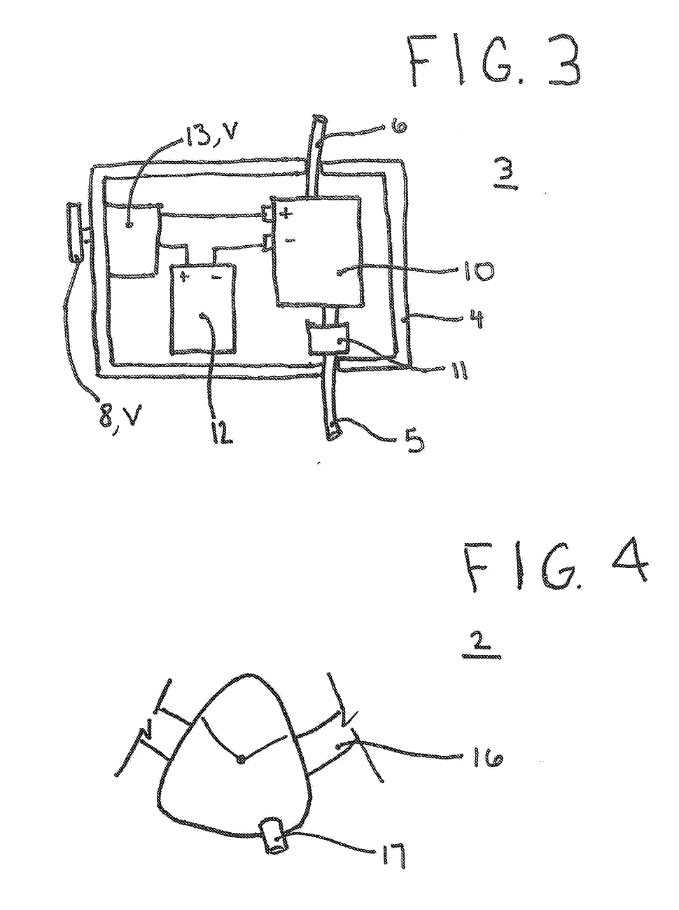

FIG. 1 is according to some embodiments a front view of a person wearing a peripheral neuropathy breathing device 20. FIG. 2 according to some embodiments includes device 20 removed from the patient of FIG. 1. Device 20 according to some embodiments includes facemask 2 which has air supplied to it by air pump apparatus 3. The air supplied to facemask 2 is air pre-warmed by the body D of the person wearing device 20. The person breathes warm air WA supplied to his body air intake port DP comprising his nose, his mouth or both. The person breathes warm air versus the cold environmental air normally breathed by the person. FIG. 3 according to some embodiments is a view of air pump apparatus 3 removed from FIG. 2 with cover 4C of apparatus housing 4 removed. FIG. 4 is according to some embodiments a view of facemask 2 removed from FIG. 2.

Referring back to FIGS. 1 thru 4 according to some embodiments includes air pump apparatus 3 having apparatus housing 4 comprising cover 4C holding air pump 10. Air pump 10 is a typical commercially available electrical air pump used to move air from one location to another. Air pump 10 according to some embodiments includes air pump input line 5 which is a flexible plastic tubular line for collecting warm air WA about the body D of a person and air pump output line 6 which is a flexible plastic tubular line for transmitting the air to facemask input port 17. According to some embodiments in-line carbon air filter 11 is optionally included to filter air entering air pump 10. Carbon air filter 11 according to some embodiments is added to prevent odors and particles from entering facemask 2. According to some embodiments battery 12 and volume control V could be at separate locations from air pump 10.

Air pump apparatus 3 according to some embodiments includes battery 12 connected in a series arrangement with air pump and volume control switch 13 such that a rotation of volume control knob 8 first turns “on”, volume control switch 13 and upon further rotation increases the voltage applied to air pump 10 to incrementally increase the warm air WA supplied to facemask 2.

According to some embodiments volume control switch 13 is a typical commercially available rheostat within an “on-off” position. According to some embodiments volume control switch 13 could be replaced by a simple “on-off” switch which would energize air pump 10 but not alter the air supplied to facemask 2. Looking at FIG. 4 facemask 2 which is according to some embodiments typically employed to supply oxygen to a person includes elastic strap 16 for holding it in place and facemask input port 17 which accepts air pump output line 6.

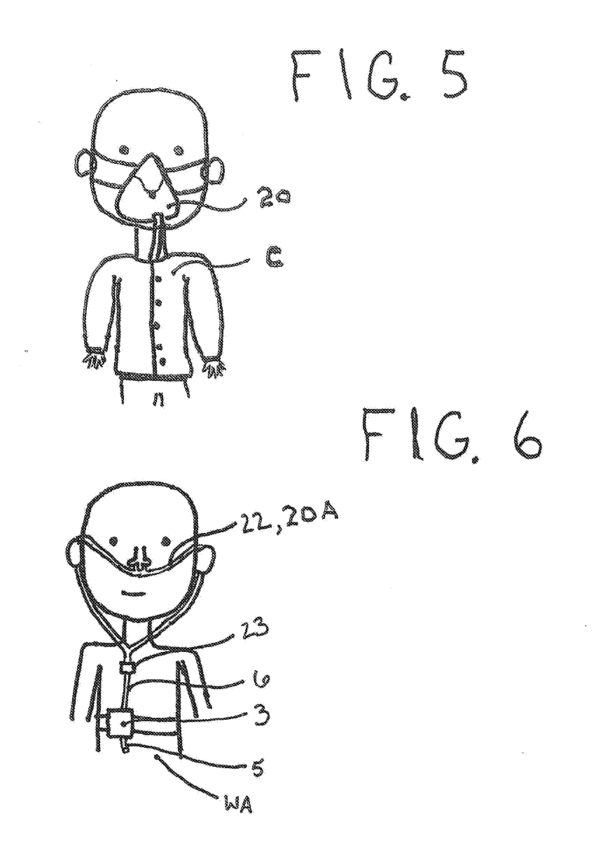

Looking back at FIG. 1 the person does not have a protective covering such as a coat or jacket. However, in use in cold weather the person would have a covering such as a coat or jacket to be later described. FIG. 5 shows the person in FIG. 1 with covering C. According to some embodiments air pump apparatus 3 would be attached to the person with belt B as shown in FIG. 1 or placed in a pocket of covering C of FIG. 5. Air pump input tube 5 according to some embodiments would be sandwiched between the coat and the body D of the person. According to some embodiments air pump input tube 5 would be up against the skin of the person. According to some embodiments air pump input tube 5 would be sandwiched between a shirt of the person and covering C. In sandwiching air pump input tube 5 beneath covering C the warm air WA sucked into input tube 5 is air that has been warmed by the body D of the person. According to some embodiments the warmed air could be taken between the head of a person and a head covering such as a hat. According to some embodiments, although a person's body D includes both the head and torso T, employing the warm air WA about the torso T of the person as shown in FIG. 1 rather than about the head of the person provides a larger and warmer supply of air. Using air pre warmed by the body D avoids a more complex device 20 which requiring a warning mechanism and a large power supply. Battery replacement and size is vital in ensuring that the person is satisfied that device 20 does not require excessive maintenance or size.

In some embodiments, in order for peripheral neuropathy breathing device 20 to be accepted by patients it must be compact and provide air at a volume according to individual's needs. Peripheral neuropathy breathing device 20 includes volume control switch 13 and volume control knob 8 to effect volume control V such that each patient can adjust the air flow to receive the exact amount of air needed. In addition, adjusting the volume of air flow controls the temperature about the body D or torso T. Control of the air about the body can be beneficial as a person can be too warm under a covering C. Increasing the air flow will reduce the temperature making the person more comfortable. Therefore, adjusting the volume of air pumped by device 20 can be used to make the person more comfortable by adjusting the volume of air supplied to the mask. The device 20 in combination with the air pump apparatus 3 can be used to make the person more comfortable by changing the temperature under the (coat) covering C.

FIG. 6, according to some embodiments, is a front view of a person wearing an alternate peripheral neuropathy breathing device 20A wearing nasal air tube 22 replacing facemask 2 from FIG. 1. Nasal air tube 22 is connected to air pump output line 6 by nasal air tube input port 23. Some patients may feel uncomfortable wearing a face mask. Nasal air tube 22 allows the person the benefits of the peripheral neuropathy breathing device 20 of FIG. 1 without making the patient or nearby persons uncomfortable due to facemask 2.

It will be readily seen by one of ordinary skill in the art that the disclosed embodiments fulfill one or more of the advantages set forth above. After reading the foregoing specification, one of ordinary skill will be able to affect various changes, substitutions of equivalents and various other embodiments as broadly disclosed herein. It is therefore intended that the protection granted hereon be limited only by the definition contained in the appended claims and equivalents thereof.

What is claimed is:

1. A peripheral neuropathy breathing device comprising:
a facemask for covering at least one of the nose or mouth of a patient, and
a battery powered air pump having an air pump input line and an air pump output line, said air pump input line disposed within an air warmed by the body of the patient, said air pump configured to be energized to bring the air from said air pump input line to said air pump output line, said air pump output line connected to said facemask and configured to bring the air to the patient, whereby the patient is able to breathe the air warmed by the body to minimize discomfort caused by breathing cold air.

2. The peripheral neuropathy breathing device according to claim 1 further comprising:
an air filter connected to said air pump input line for filtering the air.

3. The peripheral neuropathy breathing device according to claim 1 further comprising:
a volume control for adjusting a volume of the air delivered to said facemask.

4. The peripheral neuropathy breathing device according to claim 1 further comprising:
said air pump input tube is sandwiched between a body of the patient and a covering of the patient.

5. The peripheral neuropathy breathing device according to claim 1 further comprising:
said air pump input tube is sandwiched between a torso of the patient and a covering of the patient.

6. The peripheral neuropathy breathing device according to claim 1, wherein the air pump is energized by a battery.

7. A peripheral neuropathy breathing device comprising:
a nasal air tube disposed about the nose of a patient and configured to bring air to the nose of a patient, and
an air pump having an air pump input line and an air pump output line, said air pump input line disposed within an air warmed by the body of the patient, said air pump energized by a battery for bringing the air from said air pump input line to said air pump output line, said air pump output line connected to said nasal air tube, whereby the patient breathes the air warmed by the body to minimize discomfort caused by breathing cold air.

8. The peripheral neuropathy breathing device according to claim 7 further comprising:
an air filter connected to said air pump input line for filtering the air.

9. The peripheral neuropathy breathing device according to claim 7 further comprising:
a volume control for adjusting a volume of the air delivered to said nasal air tube.

10. The peripheral neuropathy breathing device according to claim 7 further comprising:
said air pump input tube is sandwiched between a body of the patient and a covering of the patient.

11. The peripheral neuropathy breathing device according to claim 7 further comprising:
said air pump input tube is sandwiched between a torso of the patient and a covering of the patient.

12. The peripheral neuropathy breathing device according to claim 7, wherein the air pump is energized by a battery.
